(19) United States
(12) Patent Application Publication
(10) Pub. No.: US 2026/0216015 A1
(43) Pub. Date: Jul. 30, 2026

(54) COMPOSITION COMPRISING PH-SENSITIVE LIPID NANOPARTICLES CONTAINING CATIONIC MATERIAL

(71) Applicant: Dong-A Pharmaceutical Co., Ltd., Seoul (KR)

(72) Inventors: Sangduk HAN, Hwaseong-si (KR); Min-Kyung KANG, Yongin-si (KR); Seungmin HYUN, Yongin-si (KR); Sungwon LEE, Yongin-si (KR); Hyoyoung PARK, Yongin-si (KR)

(21) Appl. No.: 19/141,413

(22) PCT Filed: Dec. 22, 2023
(86) PCT No.: PCT/KR2023/021496

§ 371 Date: Jun. 20, 2025

(30) Foreign Application Priority Data
Dec. 23, 2022 (KR) 10-2022-0183584

(51) Int. Cl. A61K 8/14; A61K 8/34; A61K 8/36; A61K 8/44; A61K 8/55; A61K 8/60; A61K 8/63; A61K 8/67; A61K 8/68; A61K 8/73; A61K 8/86; A61K 8/9789; A61K 9/1272; A61K 9/51; A61Q 19/00; A61Q 19/08; B82Y 5/00

(52) U.S. Cl. CPC A61K 8/14; A61K 8/342; A61K 8/345; A61K 8/361; A61K 8/447; A61K 8/553; A61K 8/608; A61K 8/63; A61K 8/671; A61K 8/68; A61K 8/73; A61K 8/86; A61K 8/9789; A61K 9/1272; A61K 9/5123; A61Q 19/005; A61Q 19/08; A61K 2800/413; A61K 2800/81; B82Y 5/00

(57) ABSTRACT

The present invention relates to pH-sensitive nanoparticles including a cationic substance which may stably encapsulate an active substance at a high content and having an excellently improved skin retention and delivery of the active ingredient when applied to skin, and a composition including the same.

### TECHNICAL FIELD

The present invention relates to pH-sensitive nanoparticles including a cationic substance, and a composition including the same. More specifically, the present invention relates to pH-sensitive nanoparticles including a cationic substance which may stably encapsulate an active substance with a high content and having an excellently improved skin retention and delivery of an active ingredient when applied to a skin, and the composition including the same.

### BACKGROUND

In cosmetics and pharmaceutical fields, there has been a demand for a development of a formulation that may stably capture various substances effective for a skin and effectively act on the skin to improve the skin condition. However, many physiologically active substances are insoluble or unstable in an aqueous phase, and often combine or react with other substances, thereby destabilizing an entire system. In order to overcome this, technology that utilizes nanometer to micrometer-sized emulsified particles to more stably and easily capture effective substances in the formulation has been recognized for its value as a core technology. As a representative example, a nanoemulsion is made of a semi-formulation using a surfactant having a specific hydrophilic-hydrophobic ratio value and then treating the same with a high-pressure emulsifier to form fine emulsified particles, and a liposome is made of a spherical or other shaped particle structure in which an effective substance is captured while forming a single or multiple membrane using a phospholipid raw material derived from plants or animals. In addition, technology has been reported for the preparation of nano-sized emulsified particles using a microemulsion formed with three phases consisting of an emulsifier, oil, and water at optimal concentrations.

These emulsified particles have a problem in that an active ingredient inside an emulsion is continuously contacting water, thereby causing denaturation due to oxidation or degradation. In addition, an emulsification membrane is very weak and unstable physically and chemically and is destroyed due to contamination by salt or charged organic or inorganic substances, and is also very weak against heat or light, and thus there is a disadvantage of being unstable for long-term storage.

Furthermore, keratin which is dead cells that are a main ingredient of stratum corneum in the skin and is a rigid support like a brick, and an intercellular lipid ingredient that is attached to the support like cement carry out various roles of a physiological protective film such as maintaining skin moisture and protecting the skin from external harmful factors, but for external agents used in cosmetics and pharmaceutical industries, it may be a major obstacle to transdermal absorption of drugs. In general, it is known in the art that drug delivery through keratin is impossible due to its very rigid structure. Therefore, there is a high demand for a carrier that may effectively transmit and permeate an active substance into the skin and retain it in sufficient amounts within the skin.

### SUMMARY

### Technical Problem

Accordingly, as a result of extensive efforts of inventors of the present invention to prepare nanoparticles in which an active ingredient may be encapsulated more efficiently and stably and having excellent particle stability, excellent delivery, permeation, and retention effects into the skin, the inventors of the present invention have confirmed that pH-sensitive nanoparticles including an active substance using a specific cationic lipid substance may have an excellent encapsulation rate of the active ingredient, particle stability, and skin penetration effect and retention of the active ingredient unlike a liposome and the like which are known as conventional skin delivery particles, thereby completing the present invention.

Accordingly, an object of the present invention is directed to providing lipid nanoparticles encapsulating an active ingredient using a cationic substance and a composition including the same.

### Technical Solution

In order to achieve the object, the present invention provides pH-sensitive lipid nanoparticles including a cationic substance, a lipid, and an active ingredient.

In addition, the present invention provides a composition including the pH-sensitive lipid nanoparticles.

### Advantageous Effects

The present invention achieves a high encapsulation rate of an active ingredient, stability of lipid nanoparticles, and skin penetration, permeability, and retention of the active ingredient by including a unique cationic substance, lipid, and the active ingredient as the lipid nanoparticles, thereby exhibiting an excellent effect for use in an external pharmaceutical composition or cosmetics for skin.

### BRIEF DESCRIPTION OF THE DRAWINGS

FIG. 1 is a view showing a result of a stability confirmation test of pH-sensitive lipid nanoparticles according to the present invention.

FIGS. 2 and 3 are views showing a result of a zeta potential confirmation test of pH-sensitive lipid nanoparticles according to the present invention.

FIGS. 4A, 4B, 4C, and 5A, 5B, and 5C are views showing a test result confirming skin permeability of the pH-sensitive lipid nanoparticles according to the present invention.

FIGS. 6A and 6B are views showing a test result confirming skin retention of the pH-sensitive lipid nanoparticles according to the present invention.

FIGS. 7A and 7B are views showing a test result confirming changes in a zeta potential of pH-sensitive lipid nanoparticles according to the present invention according to a pH.

### BEST MODE

Hereinafter, the present invention will be described in detail.

In an aspect, the present invention relates to pH-sensitive lipid nanoparticles including a cationic substance, a lipid, and an active ingredient.

The present invention includes a unique cationic lipid substance to achieve a high encapsulation rate of the active ingredient, stability of the lipid nanoparticles, and skin penetration, permeability, and retention of the active ingredient.

In a specific aspect, the cationic substance used in the present invention may be a naturally derived cationic substance or a synthetic cationic substance. Preferably, the cationic substance used in the present invention may be a lipophilic surfactant that is insoluble in water, and since an integral portion (core) of a pH-sensitive lipid nanoparticle composition according to the present invention formed by the lipophilic surfactant is a lipid, it may be preferable to use a surfactant that is highly compatible with the lipid in order to secure stable cationic particles.

The naturally derived cationic substance used in the present invention may be a cationic surfactant derived from a beet. An example of such a naturally derived cationic substance may be one or more substances selected from a group consisting of Cetearyl Betainate Mesylate, Arachidyl/Behenyl Betainate Esylate, and Stearyl/Behenyl Betainate Mesylate, but is not limited thereto. These naturally derived cationic substances have been confirmed to have a biodegradability of 94% according to the OECD 301B test method and to have a natural origin index of 0.99 according to the ISO 16128 calculation method, thereby having an advantage of ensuring safety, especially when applied to a skin.

In addition, the synthetic cationic substance used in the present invention may be one or more substances selected from a group consisting of Distearoylethyl Hydroxyethylmonium Methosulfate, Behentrimonium Methosulfate, Distearoylethyl Dimonium Chloride, and Amodimethicone, but is not limited thereto.

In the present invention, a content of the cationic substance in the lipid nanoparticles may be 0.001 to 10 wt. %, 0.01 to 5 wt. %, or 0.1 to 2 wt. % with respect to a total weight of the lipid nanoparticles, but is not limited thereto.

The lipid included in the pH-sensitive lipid nanoparticles according to the present invention may include a combination of a naturally derived oil selected from a group consisting of cocoglyceride, sunflower seed oil, caprylic/capric triglyceride and olive oil, and a polar oil selected from octyldodecanol, in addition to cetyl palmitate.

Cetyl palmitate has excellent compatibility with most lipids and emulsifiers such as a natural oil, a synthetic oil, wax, etc. and is a solid lipid with a melting point of 46 to 51° C., which may better form a stable spherical structure with appropriate rigidity inside and outside of the pH-sensitive lipid nanoparticles so that the nanoparticle form is well maintained, and accordingly, there is an advantage in that formulation stability of the composition is improved.

In the present invention, a content of the lipid in the lipid nanoparticles may be 0.1 to 40 wt. % or 5 to 30 wt. % with respect to the total weight of the lipid nanoparticles, but is not limited thereto. In addition, a content of cetyl palmitate may be 0.1 to 40 wt. % or 5 to 30 wt. % with respect to the total weight of the lipid nanoparticles, but is not limited thereto. In addition, a content of the polar oil may be 0.01 to 20 wt. % or 0.1 to 10 wt. % with respect to the total weight of the lipid nanoparticles, but is not limited thereto.

There is no limitation on the active ingredient that may be included in the pH-sensitive lipid nanoparticles according to the present invention as long as it is an active ingredient applicable to a skin, but the active ingredient may preferably be a hydrophobic ingredient or a charged ingredient. In a specific aspect, the active ingredient may be one or more ingredients selected from a group consisting of: a retinoid substance such as tretinoin, retinal, retinol, retinyl palmitate, retinyl retinoate or hydroxypinacolone retinoate; a pharmaceutically acceptable salt of heparin such as heparin or sodium heparin; taurine; ubiquinone (coenzyme Q10); hydroxydecyl ubiquinone (idebenone); tocopherol; tocopherol acetate; niacinamide; adenosine; ascorbic acid; and a derivative thereof, but is not limited thereto.

In the present invention, a content of the active ingredient in the lipid nanoparticles may be 0.001 to 20 wt. %, or 0.01 to 10 wt. % with respect to the total weight of the lipid nanoparticles, but is not limited thereto.

In the present invention, an encapsulation rate of the active ingredient in the lipid nanoparticles may be 70% or more, 75% or more, 80% or more, or 85% or more.

In the present invention, a zeta potential of the lipid nanoparticles exhibits a positive charge of 20 mV or more and 20 to 60 mV. It was confirmed that the zeta potential of the pH-sensitive lipid nanoparticles according to the present invention changes as the pH changes, and in particular, the zeta potential is converted to a +charge under a weak acidic condition of pH less than 7, thereby having an advantage of not only retaining the active ingredient in the nanoparticles well into the-charged skin, but also penetrating the active ingredient well.

As an additional aspect, the pH-sensitive lipid nanoparticles according to the present invention may further include a supplemental lipid together with the ingredients.

Such a supplemental lipid may be further included to improve particle formation and stability of the pH-sensitive lipid nanoparticles according to the present invention.

The term “lipid” as used in the present invention refers to a group of organic compounds, including an ester of a fatty acid which is insoluble in water but soluble in many organic solvents, but is not limited thereto. These are generally divided into at least three categories: (1) “simple lipid” which includes wax as well as fat and oil; (2) “complex lipid” which includes phospholipid and glycolipid; and (3) “derived lipid” such as steroid.

The supplemental lipid of the present invention may be a non-cationic lipid, for example, amphoteric lipid, phospholipid, neutral lipid, non-cationic lipid, anionic lipid, hydrophobic lipid, etc.

The amphoteric lipid refers to any suitable substance in which a hydrophobic portion of the lipid substance is oriented towards a hydrophobic phase, while a hydrophilic portion is oriented towards an aqueous phase. Hydrophilic characteristics are derived from a presence of a polar or charged group such as carbohydrate, phosphate, carboxyl, sulfato, amino, sulfhydryl, nitro, hydroxyl, and other similar groups. Hydrophobicity includes a group such as a long chain saturated and unsaturated aliphatic hydrocarbon group and a group substituted by one or more aromatic, cycloaliphatic, or heterocyclic group(s), but may be imparted by a non-polar group not limited thereto. An example of an amphoteric compound includes phospholipid, aminolipid, and sphingolipid, but is not limited thereto.

A representative example of the phospholipid includes phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine, but is not limited thereto. Other phosphorus-free compounds, such as sphingolipid, glycosphingolipid family, diacylglycerol, and β-acyloxy acid, are also in a group designated as the amphoteric lipid. Additionally, the aforementioned amphoteric lipid may be mixed with other lipids including triglyceride and sterol.

The neutral lipid refers to any of a plurality of lipid species that exist in uncharged or neutral zwitterion form at a selected pH. At a physiological pH, such a lipid includes, for example, diacylphosphatidylcholine, diacylphosphatidylethanolamine, ceramide, sphingomyelin, cephalin, cholesterol, cerebroside, and diacylglycerol.

The anionic lipid refers to any lipid that is negatively charged at the physiological pH. These lipids include phosphatidylglycerol, cardiolipin, diacylphosphatidylserine, diacylphosphatidic acid, N-dodecanoyl phosphatidylethanolamine, N-succinyl phosphatidylethanolamine, N-glutarylphosphatidylethanolamine, lysylphosphatidylglycerol, palmitoyloleyolphosphatidylglycerol (POPG), and other anionic modification groups bound to the neutral lipid, but are not limited thereto.

The hydrophobic lipid refers to a compound including a group such as a long chain saturated and unsaturated aliphatic hydrocarbon group and a group substituted by one or more aromatic, cycloaliphatic, or heterocyclic group(s), but including a non-polar group not limited thereto. A suitable example includes diacylglycerol, dialkylglycerol, N-N-dialkylamino, 1,2-diacyloxy-3-aminopropane, and 1,2-dialkyl-3-aminopropane, but is not limited thereto.

In another aspect, the non-cationic lipid may include, for example, one or more anionic lipids and/or neutral lipids. In a preferred embodiment, the non-cationic lipid includes one of the following neutral lipid ingredients: (1) cholesterol or a derivative thereof; (2) a phospholipid; or (3) a phospholipid and cholesterol or a mixture of a derivative thereof.

An example of such a cholesterol derivative includes cholestanol, cholestanone, cholestenone, coprostanol, cholesteryl-2′-hydroxyethyl ether, cholesteryl-4′-hydroxybutyl ether, and a mixture thereof, but is not limited thereto.

An example of the phospholipid includes dipalmitoylphosphatidylcholine (DPPC), distearoylphosphatidylcholine (DSPC), oleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), soybean phosphatidylcholine (SPC), sunflower seed phosphatidylcholine, and a mixture thereof, but may be the neutral lipid not limited thereto. In a preferred embodiment, the phospholipid is SPC, EPC, or a mixture thereof.

The amphoteric phospholipid may be one or more substances selected from a group consisting of phosphatidyl choline (PC) [e.g., Egg PC (EPC), Soybean PC (SPC), etc.], hydrogenated phosphatidyl choline [e.g., hydrogenated soybean phosphatidyl choline (HSPC), etc.], dioleoyl phosphatidyl choline [e.g., 1,2-dioleoyl-sn-glycero-3-phosphocholine (DOPC), etc.], dimyristoyl phosphatidyl choline [e.g., 1,2-dimyristoyl-sn-glycero-3-phosphocholine (DMPC), etc.], dipalmitoyl phosphatidyl choline [e.g., 1,2-dipalmitoyl-sn-glycero-3-phosphocholine (DPPC), etc.], distearoyl phosphatidyl choline [e.g., 1,2-distearoyl-sn-glycero-3-phosphocholine (DSPC)], myristoyl palmitoyl phosphatidyl choline [e.g., 1-myristoyl-2-palmitoyl-sn-glycero-3-phosphocholine (MPPC), etc.], myristoyl stearoyl phosphatidyl choline [e.g., 1-myristoyl-2-stearoyl-sn-glycero-3-phosphocholine (MSPC), etc.], stearoyl palmitoyl phosphatidyl choline [e.g., 1-stearoyl-2-palmitoyl-sn-glycero-3-phosphocholine (SPPC), etc.], phosphatidylethanolamine, dioleoyl phosphatidylethanolamine [e.g., 1,2-dioleoyl-sn-glycero-3-phosphoethanolamine (DOPE), etc.], dimyristoyl phosphatidylethanolamine [e.g., 1,2-dimyristoyl-sn-glycero-3-phosphoethanolamine (DMPE), etc.], dipalmitoyl phosphatidylethanolamine [e.g., 1,2-dipalmitoyl-sn-glycero-3-phosphoethanolamine (DPPE), etc.], and distearoyl phosphatidylethanolamine [e.g., 1,2-distearoyl-sn-glycero-3-phosphoethanolamine (DSPE), etc.]. Preferably, the amphoteric phospholipid may be hydrogenated soybean phosphatidyl choline (HSPC).

A preferred example of such a supplemental lipid may be one or more substances selected from a group consisting of phosphatidylcholine, ceramide NP, ceramide AP, cholesterol, and cetearyl alcohol, but is not limited thereto.

Phosphatidylcholine (PC) used in the present invention may be derived from plants (soybean, sunflower) instead of animals (egg yolk lecithin), but is not limited thereto, and may preferably be included to ensure preparation stability (particle formation).

In addition, ceramide NP is known to be an essential ingredient of a skin (stratum corneum) structure and may exhibit an effect of strengthening the skin barrier, and improving sensitive skin along with improving the formation and stability of the particles.

In addition, ceramide AP may exhibit an effect of strengthening stability and moisture retaining capacity due to its long-chain lipid structure when used together with ceramide NP along with improving the formation and stability of the particles.

In addition, cholesterol is known to be an essential ingredient of the skin (stratum corneum) structure and may strengthen skin barrier and preparation stability.

In addition, cetearyl alcohol is a higher alcohol derived from natural sources (Coconut, Palm) and as a solid lipid and may exhibit an effect of strengthening stability of the lipid nanoparticles according to the present invention.

A content of the supplemental lipid among such lipid nanoparticles may be 0.001 to 5 wt. % or 0.01 to 3 wt. % with respect to the total weight of the lipid nanoparticles, but is not limited thereto.

In addition, the pH-sensitive lipid nanoparticles according to the present invention may further include an emulsifier together with the above ingredients. A non-limiting example of such an emulsifier may include a glyceryl-based emulsifier such as glyceryl stearate, an olivate-based emulsifier such as cetearyl olivate, sorbitan olivate, and ethylhexyl olivate, a polyglyceryl stearate-based emulsifier such as polyglyceryl-10 laurate, polyglyceryl-10 stearate, and polyglyceryl-3 alkyl glucose distearate (e.g., polyglyceryl-3 methylglucose distearate), an olive-derived emulsifier such as sorbitan olivate and cetearyl olivate, a glyceryl-based emulsifier such as glyceryl stearate, a phosphate-based emulsifier such as potassium cetyl phosphate, a glucose-based emulsifier such as methyl glucose dioleate, etc., and an inulin lauryl carbamate emulsifier.

In addition, the lipid nanoparticles of the present invention may further include a solvent. Such a solvent may always include a solvent having a hydroxyl group (—OH) so that an insoluble lipid may be completely dissolved in most water to increase its efficiency. In a preferred example, it is selected and used from ethanol, 1,3-butylene glycol, 2,3-butylene glycol, propylene glycol, glycerin, 1,2-pentanediol, D-panthenol, dipropylene glycol, and mixtures of two or more thereof which are generally used in the art, and it is preferable to use a generally known usage amount depending on a content of the lipids. More preferably, it may be glycerin.

In a specific aspect, the pH-sensitive lipid nanoparticles according to the present invention may be 50 to 200 nm, 50 to 150 nm, 50 to 120 nm, 70 to 200 nm, 70 to 150 nm, 70 to 120 nm, 90 to 200 nm, 90 to 150 nm, 90 to 120 nm, 100 to 120 nm, 100 to 130 nm, 100 to 140 nm, 100 to 150 nm, or 110 nm, but is not limited thereto.

The pH-sensitive lipid nanoparticles according to the present invention exhibit the following excellent effects compared to a conventional particle substance for skin delivery, for example, a liposome.

While an interior of the conventional liposome is aqueous, an interior of the pH-sensitive lipid nanoparticles according to the present invention is oily, and thus it has an advantage of ensuring a high encapsulation rate as more oil-soluble particles are encapsulated relatively.

In a case of the conventional liposome, an exterior thereof is flexible, and thus there is a high possibility that form deformation occurs during production, whereas in a case of the pH-sensitive lipid nanoparticles according to the present invention, an exterior thereof is relatively rigid, and thus it has an advantage of being able to maintain a nanoparticle form well to secure a stable formulation.

Furthermore, when the pH-sensitive lipid nanoparticles according to the present invention are applied to the skin, they meet the skin that exhibits a low pH, and as the nanoparticles suddenly meet such a low pH, the nanoparticles are charged with a higher positive charge. In this case, there is an advantage that a negative charge and electrostatic attraction of the skin are generated more strongly so that more of the active ingredient may remain in the skin, thereby securing high skin retention and penetration ability at the same time.

In addition, the pH-sensitive lipid nanoparticles according to the present invention have an advantage that viscosity is constant and easy to homogenize due to the phase stability even when the active ingredient is the same by increasing the phase stability.

Due to the high stability of the pH-sensitive lipid nanoparticles according to the present invention, it is possible to manufacture a stabilized formulation from a cream to a low-viscosity formulation when formulating an external preparation for the skin such as cosmetics.

In another aspect, the present invention provides a pharmaceutical composition including in a form of the pH-sensitive lipid nanoparticles. Preferably, the pharmaceutical composition may be for parenteral administration, and more specifically for skin administration.

A pharmaceutically acceptable carrier may additionally include, for example, a carrier for parenteral administration. Additionally, the carrier for parenteral administration may include water, suitable oil, saline solution, aqueous glucose and glycol, etc. and may further include a stabilizing agent and a preservative. A suitable stabilizing agent may be an antioxidant such as sodium bisulfite, sodium sulfite, or ascorbic acid. A suitable preservative may be benzalkonium chloride, methyl- or propyl-paraben, and chlorobutanol.

The composition of the present invention may be administered to mammals, including humans in any method. For example, the composition may be administered orally or parenterally. A parenteral administration method may be transdermal administration. The pharmaceutical composition of the present invention may be formulated as the preparation for parenteral administration according to administration routes described above.

In a case of the preparation for parenteral administration, it may be formulated in a form of an injection, cream, lotion, ointment for external use, oil, moisturizer, gel, aerosol, and nasal inhaler by a method known in the art. A total effective amount of the composition of the present invention may be administered to a patient as a single dose or may be administered by a fractionated treatment protocol in which multiple doses are administered over a long period of time. In a dosage of the pharmaceutical composition, as an effective dose for a patient is determined by considering various factors such as a patient's age, weight, health condition, gender, severity of disease, diet, and excretion rate as well as a formulation method, administration route, and number of treatments, those having ordinary skill in the art will be able to determine an appropriate effective dose of the composition of the present invention when considering these factors. The present invention may provide a pharmaceutical composition including the pH-sensitive lipid nanoparticles for preventing, improving, or treating skin aging, wrinkles, or skin sensitivity.

In another aspect, the present invention provides a cosmetic composition including in the form of the pH-sensitive lipid nanoparticles.

An ingredient included in the cosmetic composition of the present invention includes ingredients generally used in a cosmetic composition, for example, a conventional adjuvant such as an antioxidant, stabilizing agent, dissolving agent, vitamin, pigments, and flavoring agent, and a carrier in addition to the pH-sensitive lipid nanoparticles.

The cosmetic composition of the present invention may be prepared in any formulation generally prepared in the art, and for example, may be formulated in a solution, a suspension, an emulsion, a paste, a gel, a cream, a lotion, a powder, a soap, a surfactant-containing cleansing agent, oil, a powder foundation, an emulsion foundation, a wax foundation, and a spray, but is not limited thereto. More specifically, the cosmetic composition may be prepared in a formulation of a flexible toner (skin toner), a nourishing toner (milk lotion), a nourishing cream, a massage cream, an essence, an eye cream, a cleansing cream, a cleansing foam, a cleansing water, a pack, a spray, or a powder.

Preferably, the cosmetic composition of the present invention may be a W/O cream, O/W cream, O/W essence, and hydrogel formulation and may be most preferably a hydrogel formulation, but is not limited thereto.

When the formulation of the present invention is the paste, the cream, or the gel, animal oil, vegetable oil, wax, paraffin, starch, tragacanth, cellulose derivative, polyethylene glycol, silicone, bentonite, silica, talc, or zinc oxide, etc. may be used as a carrier ingredient.

When the formulation of the present invention is the powder or the spray, lactose, talc, silica, aluminum hydroxide, calcium silicate or polyamide powder is used as the carrier ingredient, and particularly when the formulation is the spray, a propellant such as chlorofluorohydrocarbon, propane/butane, or dimethyl ether may be additionally included.

When the formulation of the present invention is the solution or the emulsion, a solvent, a dissolving agent, or an emulsifying agent is used as the carrier ingredient and for example, may be water, ethanol, isopropanol, ethyl carbonate, ethyl acetate, benzyl alcohol, benzyl benzoate, propylene glycol, 1,3-butyl glycol oil, glycerol aliphatic ester, polyethylene glycol, or fatty acid ester of sorbitan.

When the formulation of the present invention is the suspension, a liquid diluent such as water, ethanol or propylene glycol, a suspending agent such as ethoxylated isostearyl alcohol, polyoxyethylene sorbitol ester, and polyoxyethylene sorbitan ester, microcrystalline cellulose, aluminum metahydroxide, bentonite, agar, or tragacanth, etc. may be used as the carrier ingredient.

In a case where the formulation of the present invention is the surfactant-containing cleansing agent, aliphatic alcohol sulfate, aliphatic alcohol ether sulfate, sulfosuccinate monoester, isethionate, imidazolinium derivative, methyl taurate, sarcosinate, fatty acid amide ether sulfate, alkyl amidobetaine, aliphatic alcohol, fatty acid glyceride, fatty acid diethanolamide, vegetable oil, lanolin derivative, or ethoxylated glycerol fatty acid ester, etc. may be used as the carrier ingredient.

In another aspect, the present invention relates to a method of preparing the pH-sensitive lipid nanoparticles according to the present invention including the following steps of:
a) weighing water, an aqueous solvent, and an aqueous ingredient as components in an aqueous phase, and a cationic substance and a supplemental lipid as components in an oil phase, respectively, and then heating to dissolve them;
b) adding an active ingredient to the oil-phase to dissolve the same;
c) adding the aqueous-phase of the step a) to the oil-phase of the step b) and stirring to emulsify the same;
d) filtering an emulsified reactant of the step c), adjusting temperature, and injecting the same into a microfluidizer to perform high-pressure emulsification; and
e) cooling the reactant that has undergone the high-pressure emulsification in the step d).

As the aqueous solvent used in the step a), one or more solvents selected from water (purified water, etc.), alcohol (ethanol, isopropyl alcohol, polyhydric alcohols such as glycerin, propylene glycol, sorbitol, etc., and higher alcohol such as cetanol) may be used as non-limiting examples of raw materials used for manufacturing cosmetics in the art. In the step a), heating may be performed at 75 to 90° C. but is not limited thereto, and it may be adjusted to a temperature at which both the aqueous phase and the oil phase are transparently dissolved.

The aqueous ingredient refers to an ingredient that may be dissolved in water and the aqueous solvent among the ingredients included in the lipid nanoparticles and may be, for example, the emulsifier.

In the step b), after confirming the oil phase dissolved transparently, the active ingredient is added and then completely dissolved.

In the steps a) and b), in addition to the aqueous phase and oil phase ingredients, the ingredients previously included in the nanoparticles may be added to the aqueous phase or the oil phase according to their respective hydrophilicity and lipophilicity.

In the step c), the oil phase is slowly added to the aqueous phase and emulsified using a homogenizer while maintaining at 70 to 75° C. As a specific Example, although not limited thereto, homogenization may be performed under a condition of 6,000 rpm, 5 minutes to achieve emulsification.

In the step d), the emulsified preparation may be filtered using a method well known in the art. For example, filtration may be performed by sieving the emulsified preparation through a 120 mesh. Thereafter, after adjusting the temperature to 60 to 65° C., the emulsified preparation may be put into a microfludizer and the high-pressure emulsification may be performed at about 1,000 bar for 3 cycles.

As such, the pH-sensitive lipid nanoparticles according to the present invention may be prepared by cooling the high-pressure emulsified preparation to 30 to 35° C.

### MODE FOR INVENTION

### Examples

Hereinafter, the present invention will be described in more detail through Examples.

These Examples are intended to describe the present invention in more detail, and the scope of the present invention is not limited to these Examples.

### Examples 1 to 7 and Comparative Examples 1 and 2: Preparation of Lipid Nanoparticles and Liposome

The pH-sensitive lipid nanoparticles according to the present invention and a liposome was prepared according to compositions shown in Tables 1 and 2 below. More specifically, the ingredient included in the aqueous phase and the ingredient included in the oil phase among the ingredients in Tables 1 and 2 below were weighed and mixed, respectively, and then dissolved by heating at 75 to 80° C. After confirming that all of the oil phase ingredients were transparently dissolved, the active ingredient was added to the oil phase to completely dissolve. Thereafter, the oil phase in which the active ingredient was dissolved was slowly added to the aqueous phase, emulsified under a condition of homogenization of 6,000 rpm, 5 minutes while maintaining at 70 to 75° C., and emulsified for 8 minutes when preparing 1 kg or more. After filtering the emulsified preparation through a 120 mesh, the temperature was adjusted to 60 to 65° C., and then put into a high-pressure emulsifier (Microfluidics, LM20, DIXC diamond interaction chamber), and high-pressure emulsification was performed at 1,000 bar for 3 cycles.

Thereafter, the high-pressure emulsified preparation was cooled to 30 to 35° C., and then stored in a light blocked and sealed condition.

TABLE 1

| | | LNP (Lipid nanoparticles) | | | LIPOSOME |
|---|---|---|---|---|---|
| | | Comparative | Example | Example | Comparative |
| Category | Ingredient name | Example 1 | 1 | 2 | Example 2 |
| A | Water | To 100 | | | |
| | Glycerin | 5 | 5 | 5 | 20 |
| | Polyglyceryl-10 Laurate | 3 | 3 | 3 | — |
| B | Cetyl Palmitate | 20 | 20 | 20 | — |
| | Polyglyceryl-3 Methylglucose Distearate | 5 | 5 | 5 | — |
| | OLIVE OIL | 5 | 5 | 5 | 5 |
| | Distearoylethyl Hydroxyethylmonium | — | — | 0.7 | — |
| | Methosulfate | | | | |
| | Cetearyl Betainate Mesylate | — | 0.8 | — | — |
| | Cetearyl Alcohol | 1.2 | 1.2 | 1.2 | |
| C | Hydrogenated Lecithin | — | — | — | 5 |
| | Phosphatidylcholine | 0.5 | 0.5 | 0.5 | — |
| | Ceramide NP | 0.3 | 0.3 | 0.3 | 1 |
| | Ceramide AP | 0.1 | 0.1 | 0.1 | 0.1 |
| | Cholesterol | 0.3 | 0.3 | 0.3 | 0.5 |
| D | Retinol | 1 | 1 | 1 | 1 |
| Note | | | | | |

TABLE 2

| | | | | | LNP Sodium | LNP |
|---|---|---|---|---|---|---|
| | | LNP Retinoid | | | Heparin | Taurine |
| | | Example | Example | Example | Example | Example |
| Category | Ingredient name | 3 | 4 | 5 | 6 | 7 |
| A | Water | To 100 | To 100 | To 100 | To 100 | To 100 |
| | Glycerin | 5 | 5 | 5 | 5 | 5 |
| | Polyglyceryl- | 3 | 3 | 3 | 3 | 3 |
| | 10 Laurate | | | | | |
| B | Cetyl Palmitate | 20 | 20 | 20 | 20 | 20 |
| | Polyglyceryl-3 | 5 | 5 | 5 | 5 | 5 |
| | Methylglucose | | | | | |
| | Distearate | | | | | |
| | OLIVE OIL | 5 | 5 | 5 | 5 | 5 |
| | Cetearyl Betainate | 0.8 | 0.8 | 0.8 | 0.8 | 0.8 |
| | Mesylate | | | | | |
| | Cetearyl Alcohol | 1.2 | 1.2 | 1.2 | 1.2 | 1.2 |
| C | Phosphatidylcholine | 0.5 | 0.5 | 0.5 | 0.5 | 0.5 |
| | Ceramide NP | 0.3 | 0.3 | 0.3 | 0.3 | 0.3 |
| | Ceramide AP | 0.1 | 0.1 | 0.1 | 0.1 | 0.1 |
| | Cholesterol | 0.3 | 0.3 | 0.3 | 0.3 | 0.3 |
| D | Tretinoin | 1 | — | — | — | — |
| | Retinal | — | 1 | — | — | — |
| | Hydroxypinacolone | — | — | 1 | — | — |
| | Retinoate | | | | | |
| | Sodium heparin | — | — | — | 0.1 | — |
| | Taurine | — | — | — | — | 5 |
| Note | | | | | | |

### Experimental Example 1: Examination of Stability of pH-Sensitive Lipid Nanoparticles

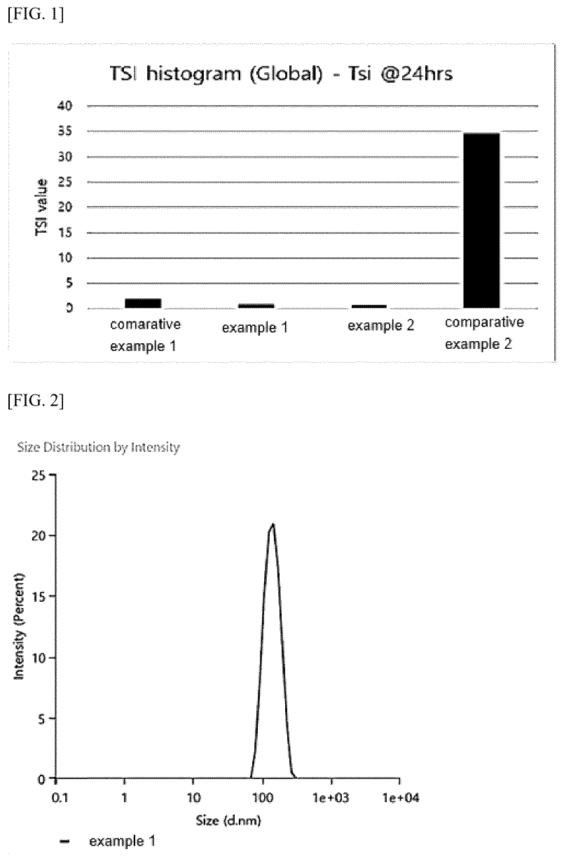

In order to confirm the stability of the pH-sensitive lipid nanoparticles according to the present invention, an experiment was conducted using the following method. Specifically, preparations of Examples 1 and 2, and preparations of Comparative Examples 1 and 2 were prepared, respectively, and the stability of the active ingredient in the nanoparticles was confirmed by liquid chromatography analysis method (HPLC, UV 325 nm (retinol), 340 nm (tretinoin) UV 368 nm (retinal), 4.6×150 mm; 5 μm column, injection volume 20 μl). The results are shown in Table 3 and FIG. 1.

TABLE 3

| | | Category 1 | | |
|---|---|---|---|---|
| | | | LNP | |
| | | LNP | Cationic | |
| | | Anionic | (Natural) | Liposome |
| | | Category 2 | | |
| | | Comparative | Example | Comparative |
| | | Example 1 | 1 | Example 2 |
| Retinol | Days | 105.03 | 102.13 | 101.97 |
| content | 14 D (25° C.) | 106.97 | 99.72 | 51.02 |
| (%) | 14 D (50° C.) | 85.40 | 89.12 | 40.39 |
| | 14 D (80° C.) | 65.93 | 69.82 | 32.77 |

| | | | |
|---|---|---|---|
| Encapsulation rate (%) | 84.88 | 95.32 | 91.45 |

As can be confirmed in Table 3 and FIG. 1, it can be confirmed that the lipid nanoparticles according to the present invention have superior properties, encapsulation rate, and stability of the active ingredient compared to the liposome.

### Experimental Example 2: Particle Size and Zeta Potential Analysis

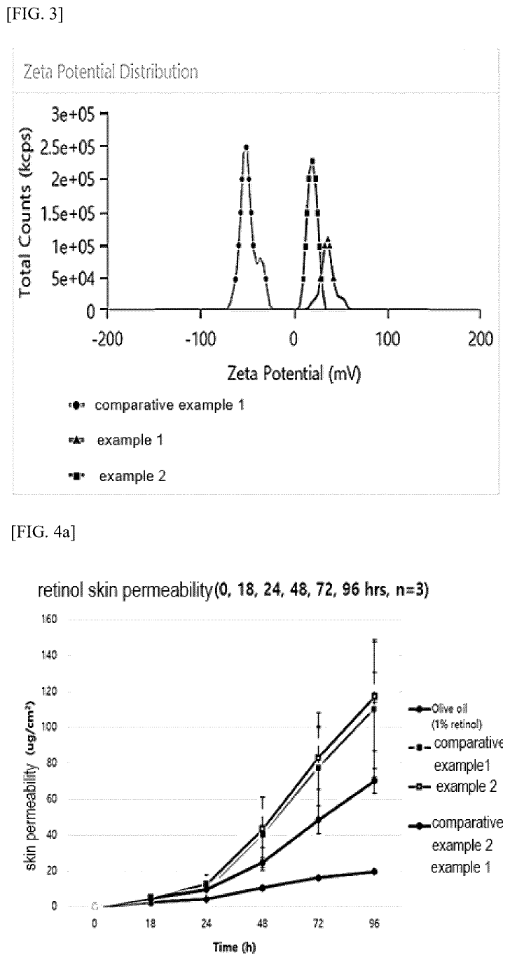

Particle size and zeta potential values were analyzed by dynamic light scattering using a Zetasizer Ultra device (Malvern Instrument, Worcestershire, UK). The results are shown in Table 4 and FIGS. 2 and 3.

TABLE 4

Category 1

| | | |
|---|---|---|
| | LNP | |
| LNP | Cationic | |
| Anionic | (Natural) | Liposome |

Category 2

| | Comparative | | Comparative |
|---|---|---|---|
| | Example 1 | Example 1 | Example 2 |
| Particle size (nm) | 127.1 | 111.4 | 110.3 |
| Zeta Potential (mV) | −48.32 | 36.44 | −35.82 |

As confirmed in Table 3, a particle size of the lipid nanoparticles according to the present invention was measured to be about 111 to 116 nm. In addition, in a case of the zeta potential, it was confirmed that it was cationic at 31 to 36 mV by application of a cationic substance.

### Experimental Example 3: Examination of Skin Permeability of pH-Sensitive Lipid Nanoparticles

In order to confirm a skin permeability of the pH-sensitive lipid nanoparticles according to the present invention, an experiment was conducted using the following method. Specifically, a skin permeation test of the lipid nanoparticles prepared by Examples and Comparative Examples was performed using the Franz diffusion cell method. The present experiment was performed using human cadaver skin (Hans Biomed, Gyeonggi-do, Republic of Korea). Stratum corneum of the cadaver skin was placed on a receptor chamber to face upward, a donor chamber was fastened, and then the present experiment was performed by the Franz diffusion cell test method. The Franz diffusion cell method is a test method that may evaluate permeation characteristics of a drug by measuring skin absorption in vitro, thereby having an advantage of allowing repeated measurements of a test substance.

Specifically, the skin was activated with PBS, the stratum corneum of the skin was placed on the receptor chamber to face upward, and the donor chamber was fastened and mounted in the Franz diffusion cell. PBS (pH 7.4) containing ethanol (50%, v/v) was used as a receptor phase. After samples of the compositions prepared in Examples and Comparative Example 1 were applied to the entire skin by 200 mg, respectively, a content of the active ingredient permeated after 0, 18, 24, and 48 hours was measured using a liquid chromatography method.

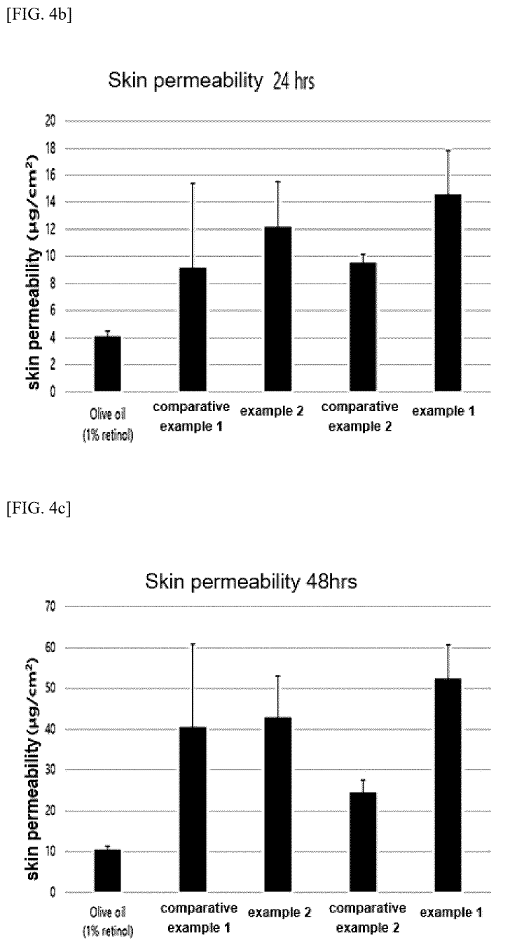

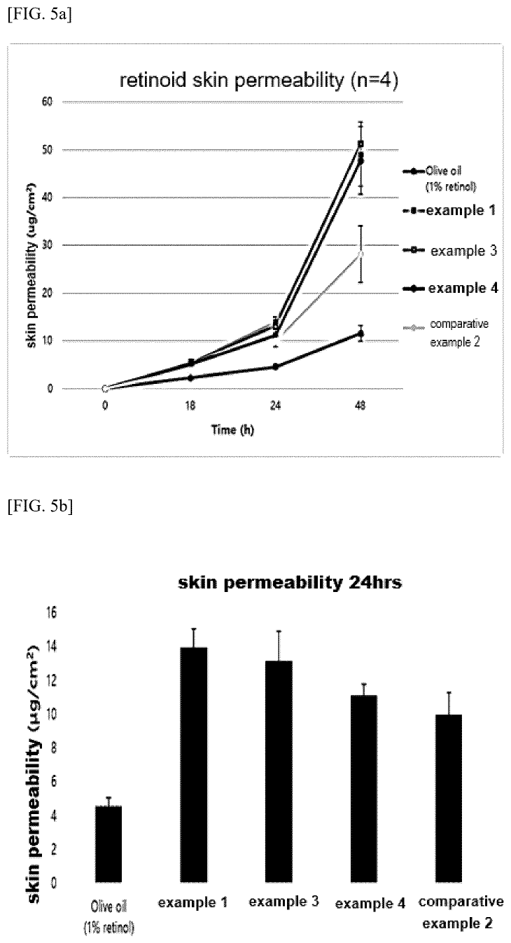

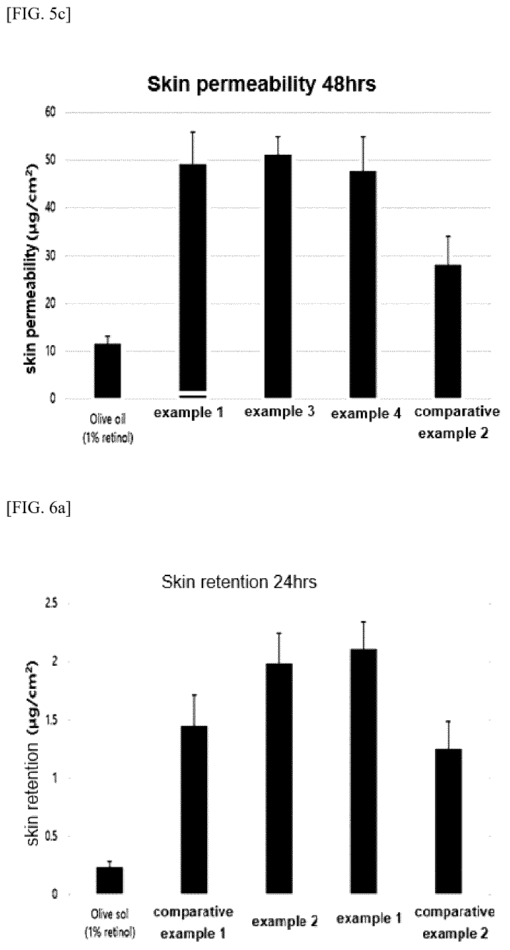

The results are shown in FIGS. 4 and 5.

As can be confirmed in the result in FIGS. 4 and 5, it can be confirmed that the lipid nanoparticles according to the present invention exhibited excellent skin permeability compared to the liposome. In particular, it can be seen that the most excellent skin permeability was obtained by using a naturally derived cationic substance as the cationic substance.

In addition, retinoids showed excellent skin permeability when encapsulated in the lipid nanoparticles according to the present invention without a significant difference depending on the type thereof.

### Experimental Example 4: Examination of Skin Retention of pH-Sensitive Lipid Nanoparticles

In order to confirm skin retention of the pH-sensitive lipid nanoparticles according to the present invention, an experiment was conducted using the following method. Specifically, the lipid nanoparticles and the liposome prepared in Examples and Comparative Examples were confirmed by the following test method.

First, cadaver skin was activated in PBS for more than 30 minutes, took out the cadaver skin, wiped with PBS, placed on a mold so that the skin surface faces upward, and then applied the lipid nanoparticles and the liposome prepared in Experimental Example 3 and 1% olive oil, respectively, and filled the bottom with PSB to prevent drying. 24 hours after the application, the lipid nanoparticles and the liposome, and the 1% olive oil were wiped off, respectively, washed with PBS three times, and moisture was minimized with Kimtech.

It was immediately immersed in a mixed solvent of dichloromethane and methanol (2:1), cut into the finest pieces with scissors, light was blocked, immersed in ice, and filtered after 30 min of Sonic process to obtain a test liquid.

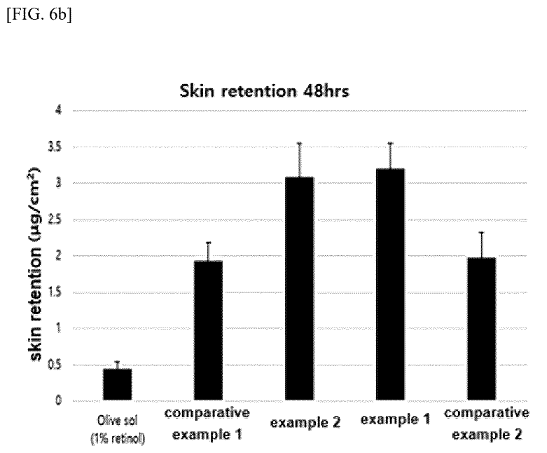

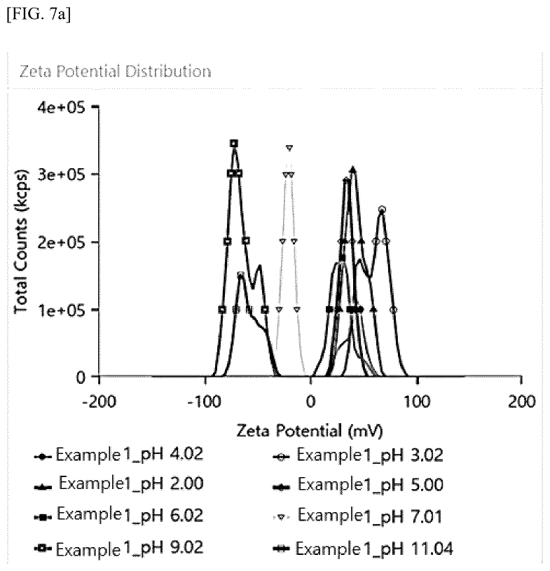

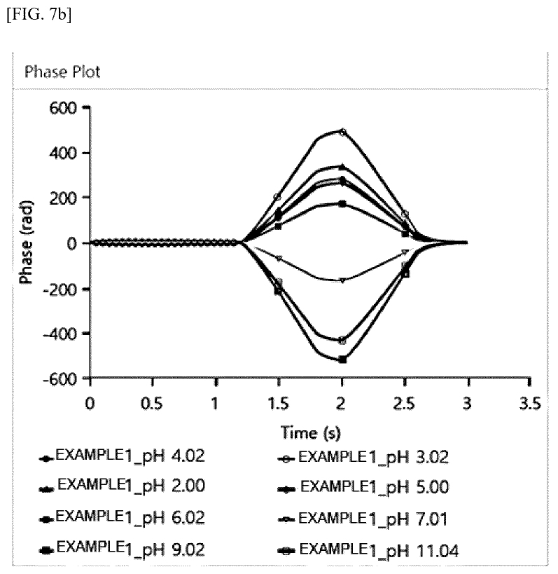

The test liquid was measured by a liquid chromatography method. The results are shown in FIG. 6.

As can be confirmed from the result in FIG. 6, it can be confirmed that the lipid nanoparticles according to the present invention exhibited excellent skin retention compared to the liposome. In particular, it can be seen that the most excellent skin permeability was obtained by using a naturally derived cationic substance as the cationic substance.

In addition, retinoids showed excellent skin retention when encapsulated in the lipid nanoparticles according to the present invention without a significant difference depending on the type thereof.

### Experimental Example 5: Examination of Zeta Potential Change of pH-Sensitive Lipid Nanoparticles

A change in the zeta potential of the pH-sensitive lipid nanoparticles according to the present invention according to a pH was confirmed. Specifically, the test was conducted in the same manner as in Experimental Example 2, but each zeta potential was measured by varying the pH as shown in Table 6. The results are shown in Table 6 and FIG. 6. As can be seen in Table 5 and FIG. 6, the lipid nanoparticles according to the present invention shows a change in the zeta potential as the pH changes, and in particular, when changing from a neutral pH to a slightly acidic pH, it can be seen that the zeta potential rapidly switches from a negative charge to a positive charge.

Accordingly, it can be seen that the charge of the lipid nanoparticles according to the present invention is changed in response to the change in the pH, which has an excellent effect on a degree to which they remain in the skin.

Specifically, the skin surface is slightly acidic and exhibits a relatively low pH. The lipid nanoparticles according to the present invention exhibits slight acidity in the formulation, but when applied to the skin surface, they meet a relatively low pH and exhibits a higher positive charge. As a result, a negative charge and electrostatic attraction of the skin are formed and left on the skin surface in greater amount and for a longer time even after washing the skin and thus the active ingredient may be effectively transmitted

TABLE 5
Zeta Potential Change According to Preparation of Example 1

| pH | Zeta Potential (mV) |
|---|---|
| 2.00 | 43.07 |
| 3.02 | 60.37 |
| 4.02 | 40.65 |
| 5.00 | 34.44 |
| 6.02 | 29.05 |
| 7.01 | −20.43 |
| 9.02 | −64.27 |
| 11.04 | −57.3 |

What is claimed is:

1. pH-sensitive lipid nanoparticles comprising a cationic substance, a lipid, and an active ingredient, wherein the active ingredient is in a form encapsulated by the cationic substance and the lipid.

2. The pH-sensitive lipid nanoparticles of claim 1, wherein the cationic substance has lipophilic properties.

3. The pH-sensitive lipid nanoparticles of claim 1, wherein the cationic substance is one or more substances selected from a group consisting of one or more naturally derived cationic substance selected from a group consisting of Cetearyl Betainate Mesylate, Arachidyl/Behenyl Betainate Esylate, and Stearyl/Behenyl Betainate Mesylate, and
one or more synthetic cationic substances selected from a group consisting of Distearoylethyl Hydroxyethylmonium Methosulfate, and Amodimethicone.

4. The pH-sensitive lipid nanoparticles of claim 1, wherein a content of the cationic substance in the lipid nanoparticles is 0.1 to 2 wt. % based on a total weight of the lipid nanoparticles.

5. The pH-sensitive lipid nanoparticles of claim 1, wherein the lipid is a combination of cetyl palmitate and an oil selected from cocoglyceride, sunflower seed oil, caprylic/capric triglyceride, olive oil, and octyldodecanol.

6. The pH-sensitive lipid nanoparticles of claim 1, wherein a content of the lipid in the lipid nanoparticles is 0.1 to 30 wt. % with respect to a total weight of the lipid nanoparticles.

7. The pH-sensitive lipid nanoparticles of claim 1, wherein the active ingredient is one or more ingredients selected from a group consisting of: a retinoid substance such as tretinoin, retinal, retinol, retinyl palmitate, retinyl retinoate or hydroxypinacolone retinoate; heparin or pharmaceutically acceptable salt of heparin such as sodium heparin; taurine; ubiquinone; hydroxydecyl ubiquinone; tocopherol; tocopherol acetate; niacinamide; adenosine; ascorbic acid; and a derivative thereof.

8. The pH-sensitive lipid nanoparticles of claim 1, wherein a content of the active ingredient in the lipid nanoparticles is 0.01 to 10 wt. % with respect to a total weight of the lipid nanoparticles.

9. The pH-sensitive lipid nanoparticles of claim 1, wherein the lipid nanoparticles exhibit a zeta potential in range of 20 to 60 mV.

10. The pH-sensitive lipid nanoparticles of claim 1, further comprising one or more supplemental lipids selected from a group consisting of phosphatidylcholine, ceramide NP, ceramide AP, cholesterol, and cetearyl alcohol.

11. The pH-sensitive lipid nanoparticles of claim 1, wherein a content of a supplemental lipid in the lipid nanoparticles is 0.01 to 3 wt. % based on a total weight of the lipid nanoparticles.

12. The pH-sensitive lipid nanoparticles of claim 1, further including one or more emulsifiers selected from a group consisting of glyceryl-based emulsifier, olivate-based emulsifier, polyglyceryl-10 laurate, and polyglyceryl-3 alkyl glucose distearate.

13. The pH-sensitive lipid nanoparticles of claim 1, wherein the pH-sensitive lipid nanoparticles have a particle size of 50 to 200 nm.

14. A pharmaceutical composition for preventing, improving, or treating skin aging, wrinkles, or skin sensitivity, comprising the pH-sensitive lipid nanoparticles of claim 1.

15. A cosmetic composition comprising the pH-sensitive lipid nanoparticles of claim 1.

16. A method of preparing the pH-sensitive lipid nanoparticles of claim 1, including:
a) weighing water, an aqueous solvent, and an aqueous ingredient as components in an aqueous phase, and a cationic substance and a supplemental lipid as components in an oil phase, respectively, and then heating to dissolve them;
b) adding an active ingredient to the oil-phase to dissolve the same;
c) adding the aqueous-phase of the step a) to the oil-phase of the step b) and stirring to emulsify the same;
d) filtering an emulsified reactant of the step c), adjusting temperature, and putting the same into a microfluidizer to perform high-pressure emulsification; and
e) cooling the reactant that has undergone the high-pressure emulsification in the step d).
